(19) United States
(12) Patent Application Publication
(10) Pub. No.: US 2015/0184250 A1
(43) Pub. Date: Jul. 2, 2015

(54) KRAS PRIMERS AND PROBES

(71) Applicant: RESPONSE GENETICS, INC., Los Angeles, CA (US)

(72) Inventor: Craig Stephens, Pasadena, CA (US)

(73) Assignee: RESPONSE GENETICS, INC., Los Angeles, CA (US)

(21) Appl. No.: 14/570,771

(22) Filed: Dec. 15, 2014

Related U.S. Application Data
(63) Continuation of application No. 13/640,416, filed on Oct. 10, 2012, now abandoned.
(60) Provisional application No. 61/323,114, filed on Apr. 12, 2010.

(51) Int. Cl. C12Q 1/68

(52) U.S. Cl. CPC C12Q 1/6886; C12Q 2600/156; C12Q 2600/106

(57) ABSTRACT

The present invention provides oligonucleotide primers or probes for the detection of a mutation of the KRAS gene. The invention also provides a method for detecting a mutation in the KRAS gene using the oligonucleotide primers or probes disclosed therein. Furthermore, the present invention encompasses a method for predicting the sensitivity of a tumor in a patient to epidermal growth factor receptor-directed chemotherapy, comprising obtaining DNA from the tumor; and determining whether there is a mutation in codon 12 and/or a mutation in codon 13 in exon 2 of the KRAS gene in the DNA using a method utilizing at least one of the oligonucleotide primers and/or probes of the present invention.

The present application claims the benefit of U.S. Provisional Patent Application No. 61/323,114 filed Apr. 12, 2010, the disclosure of which is hereby incorporated by reference.

### FIELD OF THE INVENTION

The present invention relates to PCR primers and probes for detecting KRAS mutations in DNA and methods of using the same to detect KRAS mutations and to predict the sensitivity of a cancer to epidermal growth factor receptor-directed chemotherapy.

### BACKGROUND INFORMATION

The epidermal growth factor receptor (EGFR) is a tyrosine kinase that plays an important role in cancer development. For example, over expression of EGFR was seen in more than 85% of tumors from patients with metastatic colorectal cancer (CRC). See Lee J J and Chu E, Clin Colorectal Cancer 2007; 6 Suppl 2:S42-6. Anticancer drugs targeting EGFR have been developed. Cetuximab and panitumumab are two EGFR inhibitors that have shown promising therapeutic effects in second-line use for metastatic CRC and in first-line use in combination with oxaliplatin and irinotecan-based therapies. See Lee J J and Chu E, Clin Colorectal Cancer. 2007; 6 Suppl 2:S42-6; Zhang W, et al., Ann Med. 2006; 38: 545-51. However, not all patients are responsive to cetuximab and panitumumab.

Ras genes, H-ras, K-ras (KRAS), and N-ras, encode small GTPases that are involved in the EGFR signaling pathway. A point mutation in the KRAS gene at one of the critical codons 12, 13, or 61 in exon 2 promotes tumor development. KRAS mutations occur in about 37% of colorectal adcnocarcinomas. See Brink M, et al., Carcinogenesis 2003; 24: 703-10. A strong correlation has been shown between a mutated K-ras gene and lack of response to as well as short survival from both cetuximab and panitumumab therapies. Because the presence of a KRAS mutation is highly predictive of non-response to cetuximab or panitumumab, patients with mutated KRAS should consider foregoing chemotherapies with these EGFR inhibitors.

KRAS mutations can be detected by a number of methods. For example, DNA may be extracted, e.g., by standard proteinase K digestion and phenol-chloroform extraction, from frozen tissue samples and amplified by polymerase chain reaction (PCR), wherein KRAS mutations can then be detected by sequencing of the PCR products. See Tam I Y, et al., Clin Cancer Res. 2006; 12(5): 1647-53.

KRAS mutations can also be detected with an amplification refractory mutation system PCR (ARMS PCR). ARMS PCR, also called allele-specific PCR (ASP) or PCR amplification of specific alleles (PASA), is a PCR-based method capable of detecting single base mutations. See Newton et al., Nucleic Acids Res. 1989; 17(7): 2503-16. In an ARMS PCR, the 3′ end of one of the PCR primers coincides with the target mutation. Because ARMS PCR employs a polymerase that lacks 3′ exonuclease activity (usually Taq polymerase) required for mismatch repair, ARMS PCR in principle will amplify only the DNA template with the target mutation. ARMS allows detection of a mutation solely by inspection of reaction mixtures, e.g, by agarose gel electrophoresis, because the presence of an amplified product indicates the presence of a particular mutation. See Newton et al., Nucleic Acids Res. 1989; 17(7): 2503-16; Bottema, C D, et al., Methods Enzymol. 1993; 218: 388-402.

### SUMMARY OF THE INVENTION

The present invention provides oligonucleotide primers and probes selected from:

(a) an oligonucleotide consisting of a nucleotide sequence of GTCAAGGCACTCTTGCCTAAGT (SEQ ID NO:1; hereinafter also referred to as “13ASP Reverse Primer” or “Kras38A_{—}2GT-R”) or an oligonucleotide substantially identical thereto;

(b) an oligonucleotide consisting of a nucleotide sequence of GGCCTGCTGAAAATGACTGA (SEQ ID NO:2; hereinafter also referred to as “C13 Forward Primer” or “KrasC13-F4”) or an oligonucleotide substantially identical thereto;

(c) a labeled oligonucleotide consisting of a nucleotide sequence of

6FAM-CAACTACCACAAGTTT (SEQ ID NO:3; hereinafter also referred to as “C13 Probe” or “KrasC13-Mc2”) or an oligonucleotide substantially identical thereto;

(d) an oligonucleotide consisting of a nucleotide sequence of AGGCACTCTTGCCTCCGT (SEQ ID NO:4; hereinafter also referred to as “Kras38A_{—}3TG-R”) or an oligonucleotide substantially identical thereto;

(e) an oligonucleotide consisting of a nucleotide sequence of GCCTGCTGAAAATGACTGAATAT (SEQ ID NO:5; hereinafter also referred to as” KrasC13-F″) or an oligonucleotide substantially identical thereto;

(f) a labeled oligonucleotide consisting of a nucleotide sequence of 6FAM-CTCCAACTACCACAAGTT (SEQ ID NO: 6; hereinafter also referred to as “KrasC13_Mc”) or an oligonucleotide substantially identical thereto;

(g) an oligonucleotide consisting of a nucleotide sequence of CTTGTGGTAGTTGGAGCTGGTAA (SEQ ID NO: 7; hereinafter also referred to as “13ASP Forward Primer” or “Kras38A_{—}1GA-F”) or an oligonucleotide substantially identical thereto;

(h) an oligonucleotide consisting of a nucleotide sequence of AATATAAACTTGTGGTAGTTGGAGCTTT (SEQ ID NO: 8; hereinafter also referred to as “12VAL Forward Primer”) or an oligonucleotide substantially identical thereto;

(i) an oligonucleotide consisting of a nucleotide sequence of GAATATAAACTTGTGGTAGTTGGAGCTAT (SEQ ID NO: 9; hereinafter also referred to as “KrasM35T_{—}1GA-F”) or an oligonucleotide substantially identical thereto;

(j) an oligonucleotide consisting of a nucleotide sequence of TATAAACTTGTGGTAGTTGGAGGTGT (SEQ ID NO: 10; hereinafter also referred to as “Kras35T_{—}3CG-F”) or an oligonucleotide substantially identical thereto;

(k) an oligonucleotide consisting of a nucleotide sequence of TGAAGATGTACCTATGGTCCTAGTAGGA (SEQ ID NO: 11; hereinafter also referred to as “KrasEx4 Control Forward Primer” or “KrasEx4_C-F”) or an oligonucleotide substantially identical thereto;

(l) an oligonucleotide consisting of a nucleotide sequence of GTCCTGAGCCTGTTTTGTGTCTA (SEQ ID NO: 12; hereinafter also referred to as “KrasEx4 Control Reverse Primer” or “KrasEx4_C-R”) or an oligonucleotide substantially identical thereto;

(m) a labeled oligonucleotide consisting of a nucleotide sequence of 6FAM-TAGAAGGCAAATCACA (SEQ ID NO: 13; hereinafter also referred to as “KrasEx4 Control Probe” or “KrasEx4_C-M”) or an oligonucleotide substantially identical thereto;

(n) an oligonucleotide consisting of a nucleotide sequence of TGAATATAAACTTGTGGTAGTTGGAGATA (SEQ ID NO:14; hereinafter also referred to as “12SER Forward Primer”) or an oligonucleotide substantially identical thereto;

(o) an oligonucleotide consisting of a nucleotide sequence of AATATAAACTTGTGGTAGTTGGAGGTC (SEQ ID NO:15; hereinafter also referred to as “12ARG Forward Primer”) or an oligonucleotide substantially identical thereto;

(p) an oligonucleotide consisting of a nucleotide sequence of TGAATATAAACTTGTGGTAGTTGGAGTTT (SEQ ID NO:16; hereinafter also referred to as “12CYS Forward Primer”) or an oligonucleotide substantially identical thereto;

(q) an oligonucleotide consisting of a nucleotide sequence of AAACTTGTGGTAGTTGGAGCAGA (SEQ ID NO:17; hereinafter also referred to as “12ASP Forward Primer”) or an oligonucleotide substantially identical thereto;

(r) an oligonucleotide consisting of a nucleotide sequence of AACTTGTGGTAGTTGGAGCAGC (SEQ ID NO:18; hereinafter also referred to as “12ALA Forward Primer”) or an oligonucleotide substantially identical thereto;

(s) an oligonucleotide consisting of a nucleotide sequence of CACAAAATGATTCTGAATTAGCTGTATC (SEQ ID NO:19; hereinafter also referred to as “C12 Common Reverse Primer”) or an oligonucleotide substantially identical thereto; and

(t) a labeled oligonucleotide consisting of 6FAM-TCAAGGCACTCTTGCCT (SEQ ID NO:20; hereinafter also referred to as “C12 Common Probe”) or an oligonucleotide substantially identical thereto.

One of the aspects of the present invention is a kit comprising at least one of the oligonucleotide primers and probes, (a) through (t) described above, of the invention.

The present invention also provides a method of detecting a KRAS mutation in DNA, comprising:

(1) amplifying the DNA with PCR using a thermostable DNA polymerase lacking 3′ exonuclease activity and
(I) a pair of control oligonucleotide primers for a control assay, wherein the pair of control oligonucleotide primers are for amplification of the DNA region in exon 4 of the KRAS gene, and wherein the pair of control oligonucleotide primers are KrasEx4 Control Forward Primer consisting of the nucleotide sequence represented by SEQ ID NO:11 or an oligonucleotide substantially identical thereto, and KrasEx4 Control Reverse Primer consisting of the nucleotide sequence represented by SEQ ID NO:12 or an oligonucleotide substantially identical thereto; and
(II) at least one pair of mutant oligonucleotide primers for mutation assay, wherein the at least one pair of mutant oligonucleotide primers are for amplification of the DNA region having a mutation in codon 12 and/or a mutation in codon 13 located in exon 2 of the KRAS gene, and wherein the at least one pair of mutant oligonucleotide primers are selected from
   (A) a first pair of codon 13 mutant oligonucleotide primers having
      (i) a reverse primer selected from (a) 13ASP Reverse Primer consisting of the nucleotide sequence represented by SEQ ID NO:1 (Kras38A_{—}2GT-R) or an oligonucleotide substantially identical thereto, or (b) an oligonucleotide consisting of the nucleotide sequence represented by SEQ ID NO:4 (Kras38A_{—}3TG-R) or an oligonucleotide substantially identical thereto, and
      (ii) a forward primer selected from (a) C13 Forward Primer consisting of the nucleotide sequence represented by SEQ ID NO:2 (KrasC13-F4) or an oligonucleotide substantially identical thereto, or (b) an oligonucleotide consisting of the nucleotide sequence represented by SEQ ID NO:5 (KrasC13-F) or an oligonucleotide substantially identical thereto;
   (B) a second pair of codon 13 mutant oligonucleotide primers having
      (i) a forward primer consisting of the nucleotide sequence represented by SEQ ID NO:7 (13ASP Forward Primer) or an oligonucleotide substantially identical thereto; and
      (ii) a reverse primer consisting of the nucleotide sequence represented by SEQ ID NO:19 (C12 Common Reverse Primer) or an oligonucleotide substantially identical thereto; or
   (C) at least one pair of codon 12 mutant oligonucleotide primers having
      (i) at least one forward primer selected from (a) an oligonucleotide consisting of the nucleotide sequence represented by SEQ ID NO:8 (12VAL Forward Primer) or an oligonucleotide substantially identical thereto; (b) an oligonucleotide consisting of the nucleotide sequence represented by SEQ ID NO:14 (12SER Forward Primer) or an oligonucleotide substantially identical thereto; (c) an oligonucleotide consisting of the nucleotide sequence represented by SEQ ID NO:15 (12ARG Forward Primer) or an oligonucleotide substantially identical thereto; (d) an oligonucleotide consisting of the nucleotide sequence represented by SEQ ID NO:16 (12CYS Forward Primer) or an oligonucicotidc substantially identical thereto; (c) an oligonucleotide consisting of the nucleotide sequence represented by SEQ ID NO:17 (12ASP Forward Primer) or an oligonucleotide substantially identical thereto; (f) an oligonucleotide consisting of the nucleotide sequence represented by SEQ ID NO:18 (12ALA Forward Primer) or an oligonucleotide substantially identical thereto; (g) an oligonucleotide consisting of the nucleotide sequence represented by SEQ ID NO:9 (KrasM35T_{—}1GA-F) or an oligonucleotide substantially identical thereto; or (h) an oligonucleotide consisting of the nucleotide sequence represented by SEQ ID NO:10 (Kras35T_{—}3CG-F) or an oligonucleotide substantially identical thereto; and
      (ii) an oligonucleotide reverse primer consisting of a nucleotide sequence represented by SEQ ID NO:19 (the C12 Common Reverse Primer) or an oligonucleotide substantially identical thereto;

(2) determining whether the product of step (1)(I) comprises an amplification product of the DNA region of exon 4 amplified by the pair of control oligonucleotide primers, e.g., the DNA region of exon 4 spanning from one member of the pair of control oligonucicotidc primers to the other member of the pair of control oligonucleotide primers, or spanning from a region complementary to one member of the pair of control oligonucleotide primers to a region complementary to the other member of the pair of control oligonucleotide primers, wherein the detection of the amplification product indicates the presence of the KRAS gene in the DNA; and

(3) determining whether the product of step (1)(II) comprises an amplification product of the DNA region of exon 2 amplified by the pair of mutant oligonucleotide primers, e.g., the DNA region of exon 2 spanning from one member of the at least one pair of mutant oligonucleotide primers to the other member of the at least one pair of mutant oligonucleotide primers, or spanning from a region complementary to one member of the at least one pair of mutant oligonucleotide primers to a region complementary to the other member of the at least one pair of mutant oligonucleotide primers, wherein
(a) the detection of the amplification product when at least one pair of codon 13 mutant oligonucleotide primers is used in step (1)(II) indicates the presence of a mutation in codon 13 in exon 2 of the KRAS gene in the DNA; and/or
(b) the detection of the amplification product when at least one pair of codon 12 mutant oligonucleotide primers is used in step (1)(II) indicates the presence of a mutation in codon 12 in exon 2 of the KRAS gene in the DNA.

The invention also provides a method of predicting the sensitivity of a tumor in a patient to epidermal growth factor receptor-directed chemotherapy, comprising

(1) obtaining DNA from the tumor; and
(2) determining whether there is a mutation in codon 12 and/or a mutation in codon 13 in exon 2 of the KRAS gene in the DNA using the method of the invention for detecting a KRAS mutation in DNA disclosed herein, wherein the detection of the mutation in codon 12 and/or a mutation in codon 13 predicts that the tumor has reduced sensitivity toward epidermal growth factor receptor-directed chemotherapy compared with tumors of the same type having no mutation in codon 12 and codon 13.

### DETAILED DESCRIPTION OF THE INVENTION

The presence of a mutation in the KRAS gene is highly predictive of a tumor patient's non-response to EGFR-directed chemotherapy, e.g., tumor treatments with EGFR inhibitors such as cetuximab and panitumumab. The present invention provides oligonucleotides that can be used as primers or probes in PCR to accurately and reliably detect a KRAS mutation in DNA. The present invention also provides methods of detecting a KRAS mutation in DNA using these oligonuclotides as primers or probes. The oligonucleotides disclosed herein can be made by methods known in the art, including chemical synthesis.

As used herein, the term “KRAS” refers to a Kirsten ras oncogene of, unless specified otherwise, humans. The nucleotide sequences of KRAS are well known. There are two isoforms of KRAS and the nucleotide sequences of the two isoforms can be found in GenBank under NM_{—}033360 and NM_{—}004985, the disclosures of which are herein incorporated by reference.

As used herein, the term “oligonucleotide” refers to a series of linked nucleotide residues, which oligonucleotide has a sufficient number of nucleotide residues to be used as a primer or a probe in PCR. Oligonucleotides of the invention may be modified to comprise a label, for example, a fluorescent label.

As used herein, an oligonucleotide is “substantially identical” to a subject oligonucleotide consisting of the nucleotide sequence represented by SEQ ID NO: 1 (the 13ASP Reverse Primer), 2 (the C13 Forward Primer), 4 (Kras38A_{—}3TC-R), 5 (KrasC13-F), 7 (the 13ASP Forward Primer), 8 (the 12VAL Forward Primer), 9 (KrasM35T_{—}1GA-F), 10 (Kras35G_{—}3CG-F), 11 (KrasEx4 Control Forward Primer), 12 (KrasEx4 Control Reverse Primer), 14 (the 12SER Forward Primer), 15 (the 12ARG Forward Primer), 16 (the 12CYS Forward Primer), 17 (the 12ASP Forward Primer), 18 (the 12ALA Forward Primer) or 19 (the C12 Common Reverse Primer), wherein the substantially identical oligonucleotide has at least 85%, preferably at least 90%, more preferably at least 95%, and most preferably at least 98% sequence identity with the subject oligonucleotide, and wherein there is no mismatch in the five nucleotides at the 3′ end.

The oligonucleotide substantially identical to the subject oligonucleotide consisting of the nucleotide sequence represented by SEQ ID NO: 1 (the 13ASP Reverse Primer), 2 (the C13 Forward Primer), 4 (Kras38A_{—}3TC-R), 5 (KrasC13-F), 7 (the 13ASP Forward Primer), 8 (the 12VAL Forward Primer), 9 (KrasM35T_{—}1GA-F), 10 (Kras35G_{—}3CG-F), 11 (KrasEx4 Control Forward Primer), 12 (KrasEx4 Control Reverse Primer), 14 (the 12SER Forward Primer), 15 (the 12ARG Forward Primer), 16 (the 12CYS Forward Primer), 17 (the 12ASP Forward Primer), 18 (the 12ALA Forward Primer) or 19 (the C12 Common Reverse Primer) include oligonucleotides having 1, 2 or 3 nucleotides removed from the 5′ end of the subject oligonucleotide.

The oligonucleotide substantially identical to the subject oligonucleotide consisting of the nucleotide sequence represented by SEQ ID NO: 1 (the 13ASP Reverse Primer), 2 (the C13 Forward Primer), 4 (Kras38A_{—}3TC-R), 5 (KrasC13-F), 7 (the 13ASP Forward Primer), 8 (the 12VAL Forward Primer), 9 (KrasM35T_{—}1GA-F), 10 (Kras35G_{—}3CG-F), 11 (KrasEx4 Control Forward Primer), 12 (KrasEx4 Control Reverse Primer), 14 (the 12SER Forward Primer), 15 (the 12ARG Forward Primer), 16 (the 12CYS Forward Primer), 17 (the 12ASP Forward Primer), 18 (the 12ALA Forward Primer) or 19 (the C12 Common Reverse Primer) include oligonucleotides having 1, 2 or 3 nucleotides added to the 5′ end of the subject oligonucleotide.

Examples of the oligonucleotide substantially identical to the subject oligonucleotide consisting of the nucleotide sequence represented by SEQ ID NO: 1 (the 13ASP Reverse Primer) can be CGTCAAGGCACTCTTGCCTAAGT (SEQ ID NO:21), TCGTCAAGGCACTCTTGCCTAAGT (SEQ ID NO:22) and ATCGTCAAGGCACTCTTGCCTAAGT (SEQ ID NO:23).

Examples of the oligonucleotide substantially identical to the subject oligonucleotide consisting of the nucleotide sequence represented by SEQ TD NO: 2 (the C13 Forward Primer), can be AGGCCTGCTGAAAATGACTGA (SEQ ID NO:24), AAGGCCTGCTGAAAATGACTGA (SEQ ID NO:25) and TAAGGCCTGCTGAAAATGACTGA (SEQ ID NO:26).

Examples of the oligonucleotide substantially identical to the subject oligonucleotide consisting of the nucleotide sequence represented by SEQ ID NO:4 (Kras38A_{—}3TG-R) can be AAGGCACTCTTGCCTCCGT (SEQ ID NO:27), CAAGGCACTCTTGCCTCCGT (SEQ ID NO:28) and TCAAGGCACTCTTGCCTCCGT (SEQ ID NO:29).

Examples of the oligonucleotide substantially identical to the subject oligonucleotide consisting of the nucleotide sequence represented by SEQ ID NO:5 (KrasC13-F) can be GGCCTGCTGAAAATGACTGAATAT (SEQ ID NO:30), AGGCCTGCTGAAAATGACTGAATAT (SEQ ID NO:31) and AAGGCCTGCTGAAAATGACTGAATAT (SEQ ID NO:32).

Examples of the oligonucleotide substantially identical to the subject oligonucleotide consisting of the nucleotide sequence represented by SEQ TD NO: 7 (the 13ASP Forward Primer) can be ACTTGTGGTAGTTGGAGCTGGTAA (SEQ ID NO:33), AACTTGTGGTAGTTGGAGCTGGTAA (SEQ ID NO:34) and AAACTTGTGGTAGTTGGAGCTGGTAA (SEQ ID NO:35).

Examples of the oligonucleotide substantially identical to the subject oligonucleotide consisting of the nucleotide sequence represented by SEQ ID NO:8 (the 12VAL Forward Primer) can be GAATATAAACTTGTGGTAGTTGGAGCTTT (SEQ ID NO:36), TGAATATAAACTTGTGGTAGTTGGAGCTTT (SEQ ID NO:37) and CTGAATATAAACTTGTGGTAGTTGGAGCTTT (SEQ ID NO:38).

Examples of the oligonucleotide substantially identical to the subject oligonucleotide consisting of the nucleotide sequence represented by SEQ ID NO:9 (KrasM35T_{—}1GA-F) can be TGAATATAAACTTGTGGTAGTTGGAGCTAT (SEQ ID NO:39), CTGAATATAAACTTGTGGTAGTTGGAGCTAT (SEQ ID NO:40) and ACTGAATATAAACTTGTGGTAGTTGGAGCTAT (SEQ ID NO:41).

Examples of the oligonucleotide substantially identical to the subject oligonucleotide consisting of the nucleotide sequence represented by SEQ ID NO:10 (Kras35T_{—}3CG-F) can be ATATAAACTTGTGGTAGTTGGAGGTGT (SEQ ID NO:42), AATATAAACTTGTGGTAGTTGGAGGTGT (SEQ ID NO:43) and GAATATAAACTTGTGGTAGTTGGAGGTGT (SEQ ID NO:44).

Examples of the oligonucleotide substantially identical to the subject oligonucleotide consisting of the nucleotide sequence represented by SEQ ID NO:14 (12SER Forward Primer) can be CTGAATATAAACTTGTGGTAGTTGGAGATA (SEQ ID NO:45), ACTGAATATAAACTTGTGGTAGTTGGAGATA (SEQ ID NO:46) and GACTGAATATAAACTTGTGGTAGTTGGAGATA (SEQ ID NO:47).

Examples of the oligonucleotide substantially identical to the subject oligonucleotide consisting of the nucleotide sequence represented by SEQ ID NO:15 (12ARG Forward Primer) can be GAATATAAACTTGTGGTAGTTGGAGGTC (SEQ ID NO:48), TGAATATAAACTTGTGGTAGTTGGAGGTC (SEQ ID NO:49) and CTGAATATAAACTTGTGGTAGTTGGAGGTC (SEQ ID NO:50).

Examples of the oligonucleotide substantially identical to the subject oligonucleotide consisting of the nucleotide sequence represented by SEQ ID NO:16 (12CYS Forward Primer) can be CTGAATATAAACTTGTGGTAGTTGGAGTTT (SEQ ID NO:51), ACTGAATATAAACTTGTGGTAGTTGGAGTTT (SEQ ID NO:52) and GACTGAATATAAACTTGTGGTAGTTGGAGTTT (SEQ ID NO:53).

Examples of the oligonucleotide substantially identical to the subject oligonucleotide consisting of the nucleotide sequence represented by SEQ ID NO:17 (12ASP Forward Primer) can be TAAACTTGTGGTAGTTGGAGCAGA (SEQ ID NO:54), ATAAACTTGTGGTAGTTGGAGCAGA (SEQ ID NO:55) and TATAAACTTGTGGTAGTTGGAGCAGA (SEQ ID NO:56).

Examples of the oligonucleotide substantially identical to the subject oligonucleotide consisting of the nucleotide sequence represented by SEQ ID NO:18 (12ALA Forward Primer) can be AAACTTGTGGTAGTTGGAGCAGC (SEQ ID NO:57), TAAACTTGTGGTAGTTGGAGCAGC (SEQ ID NO:58) and ATAAACTTGTGGTAGTTGGAGCAGC (SEQ ID NO:59).

Examples of the oligonucleotide substantially identical to the subject oligonucleotide consisting of the nucleotide sequence represented by SEQ ID NO:19 (C12 Common Reverse Primer) can be CCACAAAATGATTCTGAATTAGCTGTATC (SEQ ID NO:60), TCCACAAAATGATTCTGAATTAGCTGTATC (SEQ ID NO:61) and GTCCACAAAATGATTCTGAATTAGCTGTATC (SEQ ID NO:62).

As used herein, an oligonucleotide is “substantially identical” to a subject oligonucleotide consisting of a nucleotide sequence represented by SEQ ID NO: 3 (the C13 Probe), 6 (KrasC13_Mc) or 13 (the KrasEx4 Control Probe), wherein the substantially identical oligonucleotide has at least 85%, preferably at least 90%, more preferably at least 95% sequence identity with the subject oligonucleotide.

As used herein, “% sequence identity” is determined by properly aligning respective oligonucleotide segments, or their complementary strands, with appropriate considerations for nucleotide insertions and deletions. When the sequences which are compared do not have the same length, “% sequence identity” refers to the percentage of the number of identical nucleotide residues between the sequences being compared in the total number of nucleotide residues in the longer sequence.

As used herein, the term “probe” refers to an oligonucleotide of variable length, which would associate with a target DNA sequence and signal the presence and/or levels of the target sequence in a sample. For example, a probe may carry a fluorescent label and emit fluorescence under suitable conditions to signal the presence and/or levels of the target DNA sequence.

As used herein, “6FAM” refers to 6-carboxyfluorescein.

As used herein, “PCR” generally refers to polymer chain reaction, a method for amplifying a DNA sequence using a heat-stable polymerase and two oligonucleotide primers, one complementary to the (+)-strand at one end of the sequence to be amplified and the other complementary to the (−)-strand at the other end. Because the newly synthesized DNA strands can subsequently serve as additional templates, successive rounds of primer annealing, strand elongation, and dissociation produce rapid and highly specific amplification of the desired DNA sequence.

In step (1) of the method of the invention for detecting a KRAS mutation in DNA, the subject DNA can be amplified with a PCR procedure such as real time PCR.

PCR may be carried out by any of the known methods in the field. For example, the PCR may comprise preparing a mixture of the DNA to be analyzed, the oligonucleotide primers, dNTP, Mg⁺⁺, a heat-stable DNA polymerase, and a suitable buffer solution; subjecting the mixture to initial heating, e.g., to a temperature of 95° C. for 10 minutes, and then to suitable temperature cycles to amplify the DNA. For example, each temperature cycle may comprise heating the PCR mixture to 95° C. for 30 seconds and then cooling the PCR mixture to 60° C. for 1 minute. In certain embodiments, the PCR may be ARMS PCR, in which a polymerase that lacks 3′ exonuclease activity (e.g. a Taq polymerase) is used and the 3′ end of one of the primers coincides with the target KRAS mutation to be detected. A combination of ARMS PCR with other techniques, such as fluorescence labeled probes, allows detection of mutations in real time PCR reactions.

With a fluorescent labeled probe, detection of the presence of a KRAS mutation in DNA may be done using a fluorescence based real-time detection method, such as by ABI PRISM 7700 or 7900 Sequence Detection System [TaqMan®] (Applied Biosystems, Foster City, Calif.) or similar systems as described by Heid et al., (Genome Res 1996; 6:986-994) and Gibson et al. (Genome Res 1996; 6:995-1001). The output of the ABI 7700 or ABI 7900 is expressed in “Ct” or “cycle threshold,” which refers to the PCR cycle number at which the reporter fluorescence is greater than the threshold, which is an arbitrary level of fluorescence above which a signal that is detected is considered a real signal. Threshold may be chosen on the basis of the baseline variability and can be adjusted for each experiment. A higher number of target molecules in a sample generates a signal with fewer PCR cycles (lower Ct) and a lower number of target molecules in a sample generates a signal with more PCR cycles (higher Ct).

As used herein, “primer” refers to a short oligonucleotide strand that would hybridize with the beginning of a strand of the DNA template fragment to be amplified, where a DNA polymerase binds and synthesizes the new DNA strand by extending the 3′ end of the primer.

As used herein, “epidermal growth factor receptor-directed chemotherapy” or “EGFR-directed chemotherapy is chemotherapy via the administration of a substance that can impair or interfere with the signal pathway involving EGFR. The EGFR-directed chemotherapy can involve the administration of a EGFR inhibitor. Examples of the EGFR inhibitor include small-molecule tyrosine kinase inhibitors such as gefitinib and erlotinib, or anti-EGFR antibodies such as cetuximab and panitumumab.

One of the aspects of the invention is directed to a method of predicting the sensitivity of a tumor in a patient to EGFR-directed chemotherapy, comprising determining whether there is a mutation in codon 12 and/or a mutation in codon 13 in exon 2 of the KRAS gene in the DNA obtained from the tumor using the method of the invention for detecting a KRAS mutation in DNA disclosed herein. The detection of the mutation in codon 12 and/or a mutation in codon 13 predicts that the tumor has reduced sensitivity toward EGFR-directed chemotherapy compared with tumors of the same type having no mutation in codon 12 and codon 13. In some of the embodiments of the predictive method of the invention, the tumor is a lung tumor, e.g. nonsmall-cell lung cancer and lung adenocarcinoma such as lung adenocarcinoma in a patient with a smoking history, in particular, a history of heavy smoking. In some embodiments of the predictive method of the invention, the tumor is a pancreatic cancer, or preferably, colorectal cancer. If a mutation in codon 12 and/or a mutation in codon 13 of exon 2 of the KRAS gene is detected in a tumor, it is beneficial to use a tumor treatment that does not utilize EGFR-directed chemotherapy.

The invention provides the method of detecting a KRAS mutation in DNA disclosed herein. The subject DNA amplified in step (1) can be genomic DNA or cDNA obtained from a tissue of a human. A number of processes known in the art can be used to obtain the genomic DNA or cDNA. For instance, the cells in the tissue are lysed, e.g., with detergent, and the DNA is obtained by salting-out the proteins and other contaminants using ammonium or potassium acetate at a high concentration followed by centrifugation, wherein the DNA is obtained via precipitation with alcohol. In another DNA isolation method, the DNA in the lysate of the cells is precipitated with alcohol and then purified via centrifugation in a cesium chloride gradient. The DNA in the lysate of the cells can also be purified with solid-phase anion-exchange chromatography. Commercially available kits, e.g., Dynabeads DNA Direct Kit from Invitrogen or DNeasy Tissue Kit from Qiagen, can also be used to obtain genomic DNA. The genomic DNA can be DNA isolated from a formalin-fixed paraffin-embedded (FFPE) tissue with the method disclosed in U.S. Pat. Nos. 6,248,535 and 6,610,488, the disclosures of which patents are herein incorporated by reference. The method for obtaining genomic may comprise mixing a tissue sample with an organic solvent, such as phenol/chloroform/isoamyl alcohol (10:1.93:0.036), and an appropriate chaotropic agent, such as guanidinium isothiocyanate; then separating the mixture by centrifugation into three phases, a lower organic phase (containing DNA), an interphase (containing DNA), and an upper aqueous phase (containing RNA); removing the interphase; precipitating DNA in the interphase with cold ethanol or isopropanol and then centrifuging; washing the resulting DNA pellet with cold alcohol and centrifuging again; drying the DNA pellet; re-dissolving DNA in a buffer such as Tris or TE (Tris-EDTA).

The cDNA can be obtained from mRNA isolated from a tissue with reverse transcription such as using reverse-transcriptase PCR and the appropriate primers such as a poly dT oligonucleotide. For example, RT-PCR may be performed by mixing mRNA with dNTP, Bovine serum albumin (BSA), an RNAse inhibitor, random hexamers, and Moloney-Murine Leukemia Virus Reverse Transcriptase in a suitable buffer and subjecting the mixture to thermal cycles. Each thermal cycle may comprise 8 minutes at 26° C., 45 minutes at 42° C., and 5 minutes at 95° C. The mRNA can be isolated from a FFPE tissue with the method disclosed in U.S. Pat. Nos. 6,248,535 and 6,610,488. The mRNA can also be isolated from a tissue which is not an aqueous sample of a bodily fluid as disclosed in U.S. Pat. No. 6,428,963, the disclosures of which are herein incorporated by reference. The tissue from which the genomic DNA or mRNA that can be isolated may be a tumor tissue such as a colorectal cancer, e.g., metastatic colorectal cancer, pancreatic cancer, or lung cancer, e.g., lung adenocarcinoma and non-small-cell lung cancer.

An exemplary method of isolating mRNA from a paraffin-embedded tissue sample comprises: a) deparaffinizing the sample with an organic solvent, e.g. by vigorous mixing the sample with xylene followed by centrifugation at a speed sufficient to cause the tissue to pellet in the tube, usually at about 10,000 to about 20,000×g; b) rehydrating the deparaffinized sample with an aqueous solution of a lower alcohol, such as methanol, ethanol, propanols, and butanols; c) optionally homogenizing the sample using mechanical, sonic or other means of homogenization; d) heating the sample in a chaotropic solution comprising a chaotropic agent, such as guanidinium thiocyanate to a temperature in the range of about 50 to about 100° C. for about 30 to about 60 minutes; and e) recovering RNA from the chaotropic solution by any of a number of methods including extraction with an organic solvent, e.g., chloroform extraction, phenol-chloroform extraction, precipitation with ethanol or isopropanol or any other lower alcohol, by chromatography including ion exchange chromatography, size exclusion chromatography, silica gel chromatography and reversed phase chromatography, or by electrophoretic methods, including polyacrylamide gel electrophoresis and agarose gel electrophoresis. For example, RNA may be recovered as follows: 1) the sample is extracted with 2M sodium acetate at pH 4.0 and freshly prepared phenol/chloroform/isoamyl alcohol (10:1.93:0.036) by vigorous shaking for about 10 seconds and then cooling on ice for about 15 minutes; 2) the solution is centrifuged for about 7 minutes at maximum speed and the upper (aqueous) phase is transferred to a new tube; 3) the RNA is precipitated with glycogen and isopropanol for 30 minutes at −20° C.; 4) the RNA is pelleted by centrifugation for about 7 minutes in a benchtop centrifuge at maximum speed; the supernatant is decanted and discarded; and the pellet washed with about 70 to 75% ethanol; and 5) the sample is centrifuged again for 7 minutes at maximum speed. The supernatant is decanted and the pellet air dried. The pellet is then dissolved in an appropriate buffer (e.g. 50 μL, 5 mM Tris chloride, pH 8.0).

The methods of the invention are applicable to a wide range of tissue and tumor types and so can be used for assessment of prognosis for a range of cancers including breast, head and neck, lung, esophageal, colorectal, pancreatic and others. Preferably, the present methods are applied to prognosis of non-small-cell lung cancer (NSCLC) and colorectal cancer (CRC). A mutation in codon 12 and/or codon 13 in exon 2 of the KRAS gene in a cancer indicates a reduced sensitivity of the cancer to EGFR-directed chemotherapy. The cancer can be lung cancer such as lung adenocarcinoma and NSCLC, and colorectal cancer.

The DNA polymerase used in step (1) of the method of the invention for detecting a KRAS mutation in DNA is a thermostable DNA polymerase that lacks 3′ exonuclease activity. Due to the lack of 3′ exonuclease activity, the DNA polymerase will have difficulty in extending an oligonucleotide primer having a mismatch with the DNA to be amplified at the 3′ end of the primer. Examples of the thermostable DNA polymerase lacking 3′ exonuclease activity include thermostable Bst DNA polymerase I isolated from Bacillus stearothermophilus (Alitotta et al., Genetic Analysis: Biomolecular Engineering 1996, vol. 12, pp. 185-195); IsoTherm DNA polymerase (available from Epicentre Technologies, Madison, Wisconin); T7 DNA polymerase having the 3′ to 5′ exonuclease activity removed via oxidation of the amino acid residues essential for the exonuclease activity (Sequenase Vertion 1) or genetically by deleting 28 amino acids essential for the 3′ to 5′ exonuclease activity (Sequenase Version 2); Vent_{R}(exo⁻) DNA polymerase; and, preferably, Taq polymerase.

In step (2) of the method of the invention for detecting a KRAS mutation in DNA, whether the product of step (1)(I) comprises the amplification product of the DNA region of exon 4 spanning from one member of the pair of control oligonucleotide primers to the other member of the pair of control oligonucleotide primers, or spanning from a region complementary to one member of the pair of control oligonucleotide primers to a region complementary to the other member of the pair of control oligonucleotide primers, can be determined with an appropriate procedure known in the art. For instance, whether the product of step (1)(I) comprises the amplification product of the DNA region of exon 4 can be determined with DNA sequencing of the product of step (1)(I) and comparing the obtained nucleotide sequence with the nucleotide sequence of exon 4 of the KRAS gene spanning from one member of the pair of control oligonucleotide primers to the other member of the pair of control oligonucleotide primers.

Alternatively, in step (2) of the method of the invention for detecting a KRAS mutation in DNA, whether the product of step (1)(I) comprises the amplification product of the DNA region of exon 4 spanning from one member of the pair of control oligonucleotide primers to the other member of the pair of control oligonucleotide primers, or spanning from a region complementary to one member of the pair of control oligonucleotide primers to a region complementary to the other member of the pair of control oligonucleotide primers, can be determined by the use of an oligonucleotide probe for an appropriate segment of the exon 4 sequence of the KRAS gene spanning from one member of the pair of control oligonucleotide primers to the other member of the pair of control oligonucleotide primers. For instance, step (2) of the method can comprise mixing the PCR product of step (1)(I) with an oligonucleotide probe specific for a DNA region of exon 4 located between (a) the KrasEx4 Control Forward Primer and a region complementary to the KrasEx4 Control Reverse Primer, or (b) the KrasEx4 Control Reverse Primer and a region complementary to the KrasEx4 Control Forward Primer, wherein hybridization of the oligonucleotide probe with the DNA region of exon 4 shows that the product of step (1)(I) comprises the amplification product of the DNA region of exon 4 indicating that the subject DNA comprises the KRAS gene. An example of the oligonucleotide probe is KrasEx4 Control Probe consisting of the nucleotide sequence of SEQ ID NO:13, or an oligonucleotide substantially identical thereto.

Similarly, in step (3) of the method of the invention for detecting a KRAS mutation in DNA, whether the product of step (1)(II) comprises the amplification product of the DNA region of exon 2 containing mutated codon 12 and/or mutated codon 13, wherein the amplification product spans from one member of the at least one pair of mutant oligonucleotide primers to the other member of the at least one pair of mutant oligonucleotide primers, or spans from a region complementary to one member of the at least one pair of mutant oligonucleotide primers to a region complementary to the other member of the at least one pair of mutant oligonucleotide primers, can be determined with an appropriate procedure known in the art. For instance, whether the product of step (1)(II) comprises the amplification product of the DNA region containing mutated codon 12 and/or mutated codon 13 in exon 2 can be determined with DNA sequencing of the product of step (1)(II) and comparing the obtained nucleotide sequence with the nucleotide sequence of exon 2 of the KRAS gene spanning from one member of the at least one pair of mutant oligonucleotide primers to the other member of the at least one pair of mutant oligonucleotide primers.

Alternatively, in step (3) of the method of the invention for detecting a KRAS mutation in DNA, whether the product of step (1)(II) comprises the amplification product of the DNA region of exon 2 containing mutated codon 12 and/or mutated codon 13, wherein the amplification product spans from one member of the at least one pair of mutant oligonucleotide primers to the other member of the at least one pair of mutant oligonucleotide primers, or spans from a region complementary to one member of the at least one pair of mutant oligonucleotide primers to a region complementary to the other member of the at least one pair of mutant oligonucleotide primers, can be determined by the use of an oligonucleotide probe for an appropriate segment of the exon 2 sequence of the KRAS gene spanning from one member of the at least one pair of mutant oligonucleotide primers to the other member of the at least one pair of mutant oligonucleotide primers.

For instance, when the first pair of codon 13 mutant oligonucleotide primers are used in step (1)(II) of the method of the invention for detecting a KRAS mutation, as recited in step (1)(II)(A), step (3) of the method can comprise mixing the PCR product of step (1)(II) and an oligonucleotide probe specific for a DNA region of exon 2 located between (a) the reverse primer recited in step (1)(II)(A)(i) and a region complementary to the forward primer recited in step (1)(II)(A)(ii), or (b) the forward primer recited in step (1)((II)(A)(ii) and a region complementary to the reverse primer recited in step (1)(11)(A)(i), wherein hybridization of the oligonucleotide probe with the DNA region of exon 2 shows that the product of step (1)(II) comprises the amplification product of the DNA region containing codon 13 of exon 2 indicating that the subject DNA comprises a mutation in codon 13 of exon 2 of the KRAS gene. Examples of the oligonucleotide probe are (a) C13 Probe consisting of the nucleotide sequence of SEQ ID NO:3, or an oligonucleotide substantially identical thereto, and (b) KrasC13_Mc consisting of the nucleotide sequence represented by SEQ ID NO:6, or an oligonucleotide substantially identical thereto.

For instance, when the second pair of codon 13 mutant oligonucleotide primers are used in step (1)(II) of the method of the invention for detecting a KRAS mutation, as recited in step (1)(II)(B), step (3) of the method can comprise mixing the PCR product of step (1)(II) and an oligonucleotide probe specific for a DNA region of exon 2 located between (a) the forward primer recited in step (1)(II)(B)(i) and a region complementary to the reverse primer recited in step (1)(II)(B)(ii), or (b) the reverse primer recited in step (1)((II)(B)(ii) and a region complementary to the forward primer recited in step (1)(II)(B)(i), wherein hybridization of the oligonucleotide probe with the DNA region of exon 2 shows that the product of step (1)(II) comprises the amplification product of the DNA region containing codon 13 of exon 2 indicating that the subject DNA comprises a mutation in codon 13 of exon 2 of the KRAS gene. An example of the oligonucleotide probe is C12 Common Probe consisting of the nucleotide sequence of SEQ ID NO:20, or an oligonucleotide substantially identical thereto.

For instance, when the at least one pair of codon 12 mutant oligonucleotide primers are used in step (1)(II) of the method of the invention for detecting a KRAS mutation, as recited in step (1)(II)(C), step (3) of the method can comprise mixing the PCR product of step (1)(II) and an oligonucleotide probe specific for a DNA region of exon 2 located between (a) the at least one forward primer recited in step (1)(II)(C)(i) and a region complementary to the at least one reverse primer recited in step (1)(II)(C)(ii), or (b) the at least one reverse primer recited in step (1)((IT)(C)(ii) and a region complementary to the at least one forward primer recited in step (1)(II)(C)(i), wherein hybridization of the oligonucleotide probe with the DNA region of exon 2 shows that the product of step (1)(II) comprises the amplification product of the DNA region containing codon 12 of exon 2 indicating that the subject DNA comprises a mutation in codon 12 of exon 2 of the KRAS gene. An example of the oligonucleotide probe is C12 Common Probe consisting of the nucleotide sequence of SEQ ID NO:20, or an oligonucleotide substantially identical thereto.

In some of the embodiments of the method of the invention for detecting KRAS mutation of DNA, step (1)(II) uses the at least one pair of mutant oligonucleotide primers comprising
(A) the first pair of codon 13 mutant oligonucleotide primers having
   (i) 13ASP Reverse Primer, as the reverse primer, consisting of the nucleotide sequence represented by SEQ ID NO:1 or an oligonucleotide substantially identical thereto, and
   (ii) C13 Forward Primer, as the forward primer, consisting of the nucleotide sequence represented by SEQ ID NO:2 or an oligonucleotide substantially identical thereto; or
(B) the second pair of codon 13 mutant oligonucleotide primers having
   (i) 13ASP Forward Primer, as the forward primer, consisting of the nucleotide sequence represented by SEQ ID NO:7 or an oligonucleotide substantially identical thereto; and
   (ii) C12 Common Reverse Primer, as the reverse primer, consisting of the nucleotide sequence represented by SEQ ID NO:19 or an oligonucleotide substantially identical thereto.

In some of the embodiments of the method of the invention for detecting KRAS mutation of DNA, step (1)(11) uses the at least one pair of mutant oligonucleotide primers comprising
(A) the first pair of codon 13 mutant oligonucleotide primers having
   (i) 13ASP Reverse Primer, as the reverse primer, consisting of the nucleotide sequence represented by SEQ ID NO:1 or an oligonucleotide substantially identical thereto, and
   (ii) C13 Forward Primer, as the forward primer, consisting of the nucleotide sequence represented by SEQ ID NO:2 or an oligonucleotide substantially identical thereto; and
(B) the second pair of codon 13 mutant oligonucleotide primers having
   (i) 13ASP Forward Primer, as the forward primer, consisting of the nucleotide sequence represented by SEQ ID NO:7 or an oligonucleotide substantially identical thereto; and
   (ii) C12 Common Reverse Primer, as the reverse primer, consisting of the nucleotide sequence represented by SEQ ID NO:19 or an oligonucleotide substantially identical thereto.

In some of the embodiments of the method of the invention for detecting KRAS mutation of DNA, step (1)(II) uses the at least one pair of mutant oligonucleotide primers comprising
(C) at least one pair of the codon 12 mutant oligonucleotide primers having
   (i) the following primers as forward primers: (a) an oligonucleotide consisting of the nucleotide sequence represented by SEQ ID NO:8 (12VAL Forward Primer) or an oligonucleotide substantially identical thereto; (b) an oligonucleotide consisting of the nucleotide sequence represented by SEQ ID NO:14 (12SER Forward Primer) or an oligonucleotide substantially identical thereto; (c) an oligonucleotide consisting of the nucleotide sequence represented by SEQ ID NO:15 (12ARG Forward Primer) or an oligonucleotide substantially identical thereto; (d) an oligonucleotide consisting of the nucleotide sequence represented by SEQ ID NO:16 (12CYS Forward Primer) or an oligonucleotide substantially identical thereto; (e) an oligonucleotide consisting of the nucleotide sequence represented by SEQ ID NO:17 (12ASP Forward Primer) or an oligonucleotide substantially identical thereto; (f) an oligonucleotide consisting of the nucleotide sequence represented by SEQ ID NO:18 (12ALA Forward Primer) or an oligonucleotide substantially identical thereto; (g) an oligonucleotide consisting of the nucleotide sequence represented by SEQ ID NO:9 (KrasM35T_{—}1GA-F) or an oligonucleotide substantially identical thereto; or (h) an oligonucleotide consisting of the nucleotide sequence represented by SEQ ID NO:10 (Kras35T_{—}3CG-F) or an oligonucleotide substantially identical thereto; and
   (ii) an oligonucleotide reverse primer consisting of a nucleotide sequence represented by SEQ ID NO:19 (the C12 Common Reverse Primer) or an oligonucleotide substantially identical thereto.

In some of the embodiments of the method of the invention for detecting KRAS mutation of DNA, step (1)(II) uses the at least one pair of mutant oligonucleotide primers comprising
(C) codon 12 mutant oligonucleotide primers having
   (i) the following primers as forward primers: (a) an oligonucleotide consisting of the nucleotide sequence represented by SEQ ID NO:8 (12VAL Forward Primer) or an oligonucleotide substantially identical thereto; (b) an oligonucleotide consisting of the nucleotide sequence represented by SEQ ID NO:14 (12SER Forward Primer) or an oligonucleotide substantially identical thereto; (c) an oligonucleotide consisting of the nucleotide sequence represented by SEQ ID NO:15 (12ARG Forward Primer) or an oligonucleotide substantially identical thereto; (d) an oligonucleotide consisting of the nucleotide sequence represented by SEQ ID NO:16 (12CYS Forward Primer) or an oligonucleotide substantially identical thereto; (e) an oligonucleotide consisting of the nucleotide sequence represented by SEQ ID NO:17 (12ASP Forward Primer) or an oligonucleotide substantially identical thereto; (f) an oligonucleotide consisting of the nucleotide sequence represented by SEQ ID NO:18 (12ALA Forward Primer) or an oligonucleotide substantially identical thereto; (g) an oligonucleotide consisting of the nucleotide sequence represented by SEQ ID NO:9 (KrasM35T_{—}1GA-F) or an oligonucleotide substantially identical thereto; and (h) an oligonucleotide consisting of the nucleotide sequence represented by SEQ ID NO: 10 (Kras35T_{—}3CG-F) or an oligonucleotide substantially identical thereto; and
   (ii) an oligonucleotide reverse primer consisting of a nucleotide sequence represented by SEQ ID NO:19 (the C12 Common Reverse Primer) or an oligonucleotide substantially identical thereto.

In some embodiments of the method of the invention for detecting KRAS mutation of DNA, the at least one pair of mutant oligonucleotide primers used in step (1)(II) comprises
(A) the first pair of codon 13 mutant oligonucleotide primers having
   (i) 13ASP Reverse Primer, as the reverse primer, consisting of the nucleotide sequence represented by SEQ ID NO:1 or an oligonucleotide substantially identical thereto, and
   (ii) C13 Forward Primer, as the forward primer, consisting of the nucleotide sequence represented by SEQ ID NO:2 or an oligonucleotide substantially identical thereto;
(B) the second pair of codon 13 mutant oligonucleotide primers having
   (i) 13ASP Forward Primer, as the forward primer, consisting of the nucleotide sequence represented by SEQ ID NO:7 or an oligonucleotide substantially identical thereto; and
   (ii) C12 Common Reverse Primer, as the reverse primer, consisting of the nucleotide sequence represented by SEQ ID NO:19 or an oligonucleotide substantially identical thereto; and
(C) the codon 12 mutant oligonucleotide primers having
   (i) the following primers as forward primers: (a) an oligonucleotide consisting of the nucleotide sequence represented by SEQ ID NO:8 (12VAL Forward Primer) or an oligonucleotide substantially identical thereto; (b) an oligonucleotide consisting of the nucleotide sequence represented by SEQ ID NO:14 (12SER Forward Primer) or an oligonucleotide substantially identical thereto; (c) an oligonucleotide consisting of the nucleotide sequence represented by SEQ ID NO:15 (12ARG Forward Primer) or an oligonucleotide substantially identical thereto; (d) an oligonucleotide consisting of the nucleotide sequence represented by SEQ ID NO:16 (12CYS Forward Primer) or an oligonucleotide substantially identical thereto; (e) an oligonucleotide consisting of the nucleotide sequence represented by SEQ ID NO:17 (12ASP Forward Primer) or an oligonucleotide substantially identical thereto; (f) an oligonucleotide consisting of the nucleotide sequence represented by SEQ ID NO:18 (12ALA Forward Primer) or an oligonucleotide substantially identical thereto; (g) an oligonucleotide consisting of the nucleotide sequence represented by SEQ ID NO:9 (KrasM35T_{—}1GA-F) or an oligonucleotide substantially identical thereto; and (h) an oligonucleotide consisting of the nucleotide sequence represented by SEQ ID NO:10 (Kras35T_{—}3CG-F) or an oligonucleotide substantially identical thereto; and
   (ii) the oligonucleotide reverse primer consisting of a nucleotide sequence represented by SEQ ID NO:19 (the C12 Common Reverse Primer) or an oligonucleotide substantially identical thereto.

In some of the embodiments of the method of the invention for detecting a KRAS mutation in DNA, in step (1) the DNA, the pair of control oligonucleotide primers and the at least one pair of mutant oligonucleotide primers are mixed with Reaction Mix A, which is a mixture of TaqMan 1000 Reaction Gold/Buffer A Pack from Applied Biosystems and 100 mM total dNTP, which can be obtained from Applied Biosystems or GE Healthcare.

In some of the embodiments of the method of the invention for detecting a KRAS mutation in the subject DNA, the method is also applied to a DNA-negative control also referred to as the no-template control (NTC) in addition to the subject DNA. The method is applied to the subject DNA, and a separate run of the method is also applied substantially simultaneously to the NTC in a parallel fashion wherein in the NTC a liquid sample containing no DNA, instead of the subject DNA, is used in step (1). In other words, the liquid sample containing no DNA is subject to the PCR using the thermostable DNA polymerase lacking 3′ exonuclease activity and the primers recited in step (1). The method should result in no amplification products in steps (2) and (3), when the liquid sample containing no DNA is used instead of the subject DNA. The liquid sample containing no DNA should be the same liquid medium, e.g., an appropriate buffer such as 5 mM Tris, pH 8.0, used to hold the subject DNA except that there is no DNA in the liquid medium. For instance, if the subject DNA is obtained from a FFPE tissue, the liquid sample containing no DNA for the DNA-negative control or NTC run can be a 5 mM Tris buffer, pH 8.0, containing guanidinium isothiocyanate but no DNA.

In certain embodiments of the method of the invention for detecting a KRAS mutation in DNA, real time PCR may be used, wherein the real time PCR can be conducted with the following cycling parameters:

Stage 1: 50° C. for 15 seconds for one cycle;

Stage 2: 95° C. for 10 minutes for one cycle; and

Stage 3: 95° C. for 15 seconds and 60° C. for 1 minute for 42 cycles.

In some of the embodiments of the method of the invention for detecting a KRAS mutation in DNA using real time PCR, the DNA is amplified with PCR in the control assay and the mutation assay (in step (1)(I) and step (1)(II), respectively, of the method for detecting a KRAS mutation of the invention) and the amplification products can be identified using fluorescent labeled oligonucleotide probes, and then the method further comprises determining the values of Mutation Ct, Control Ct, and delta Ct, and determining the presence of a KRAS mutation in the DNA by comparing the delta Ct value with a predetermined delta Ct value disclosed in Table 2.

As used herein, “Mutation Ct” refers to the Ct for the mutation assay wherein the DNA is amplified with at least one pair of mutant oligonucleotide primers as described in step (1)(II) of the method for detecting a KRAS mutation of the invention, wherein the at least one pair of mutant oligonucleotide primers is specific for a mutation in codon 12 or 13 of exon 2. The “Mutation Ct” is the PCR cycle number at which the reporter fluorescence from the mutation assay is greater than a threshold. The term “Control Ct”, as used herein, refers to the Ct for the control assay wherein the DNA is amplified with a pair of control oligonucleotide primers as described in step (1)(I) of the method for detecting a KRAS mutation of the invention. The Control Ct is the PCR cycle number at which the reporter fluorescence from the control assay is greater than a threshold. The threshold can be set at a point to provide a Ct value between 27.0-29.0 for the control assay of gDNA with the use of KrasEx4 Forward Control and KrasEx4 Reverse Control primers in step (1)(I), wherein the gDNA (#G3041) obtainable commercially from Promega is used in place of the subject or test DNA in step (1).

As used herein, “delta Ct (ΔCt)” refers to the difference between Mutation Ct and Control Ct, i.e.,

ΔCt=[Mutation Ct]−[Control Ct].

In some of the embodiments of the method of the invention for detecting a KRAS mutation in DNA, the method is applied to a test sample of subject DNA and separately the method can also be applied to a DNA-negative control (the NTC) sample in a parallel fashion. Each of the NTC sample and the test sample of the subject DNA can be run in duplicate, and the average value of the mutation Ct and the average value of the control Ct for the duplicate runs of each of the NTC sample and the test sample are calculated, and from the average mutation Ct and the average control Ct the delta Ct for each of the NTC sample and the test sample are also calculated. When real time PCR is used on the test sample of the subject DNA, along with the parallel run on the NTC, the method should give average Ct values that are greater than or equal to the acceptance criteria listed in Table 1 for the NTC.

TABLE 1
Acceptance Criteria of Average Ct for NTC Sample

| Primer Used | NTC |
|---|---|
| KrasEx4 | 37 |
| 12SER Forward (AGT) | 40 |
| 12ARG Forward (CGT) | 40 |
| 12CYS Forward (TGT) | 40 |
| 12ASP Forward (GAT) | 40 |
| 12ALA Forward (GCT) | 40 |
| 12VAL Forward (GTT) | 40 |
| 13ASP Reverse (GAC_R) | 40 |

If the average Ct values for the NTC are greater than or equal to the Ct acceptance criteria listed in Table 1, in these embodiments of the method of the invention for detecting a KRAS mutation in DNA, the results of the method on the test sample of the subject DNA are considered acceptable if the average Ct value for the test sample of the subject DNA is less than or equal to the maximum Ct values listed in Table 2 for the specific primers used.

TABLE 2
Ct Acceptance Criteria for Test Sample

| Primer Used | Maximum Ct | Maximum ΔCt |
|---|---|---|
| KrasEx4 | 30 | N/A |
| 12SER Forward | 37.4 | 6.5 |
| 12ARG Forward | 35.9 | 6.6 |
| 12CYS Forward | 36.8 | 6.7 |
| 12ASP Forward | 36.0 | 6.8 |
| 12ALA Forward | 37.7 | 6.6 |
| 12VAL Forward | 38.4 | 6.7 |
| 13ASP Reverse | 36.7 | 6.7 |

In these embodiments, when the delta Ct value is lower than the maximum delta Ct value listed in Table 2 for the specific mutant primer used, a KRAS mutation is determined to be present in the test sample of the subject DNA in the codon corresponding to the specific mutant primer used.

I claim:

1. An oligonucleotide selected from:
(a) an oligonucleotide consisting of a nucleotide sequence of GTCAAGGCACTCTTGCCTAAGT (SEQ ID NO:1) or an oligonucleotide substantially identical thereto;
(b) an oligonucleotide consisting of a nucleotide sequence of GGCCTGCTGAAAATGACTGA (SEQ ID NO:2) or an oligonucleotide substantially identical thereto;
(c) a labeled oligonucleotide consisting of a nucleotide sequence of
6FAM-CAACTACCACAAGTTT (SEQ ID NO:3) or an oligonucleotide substantially identical thereto;
(d) an oligonucleotide consisting of a nucleotide sequence of AGGCACTCTTGCCTCCGT (SEQ ID NO:4) or an oligonucleotide substantially identical thereto;
(e) an oligonucleotide consisting of a nucleotide sequence of GCCTGCTGAAAATGACTGAATAT (SEQ ID NO:5) or an oligonucleotide substantially identical thereto;
(f) a labeled oligonucleotide consisting of a nucleotide sequence of 6FAM-CTCCAACTACCACAAGTT (SEQ ID NO: 6) or an oligonucleotide substantially identical thereto;
(g) an oligonucleotide consisting of a nucleotide sequence of CTTGTGGTAGTTGGAGCTGGTAA (SEQ ID NO: 7) or an oligonucleotide substantially identical thereto;
(h) an oligonucleotide consisting of a nucleotide sequence of AATATAAACTTGTGGTAGTTGGAGCTTT (SEQ ID NO: 8) or an oligonucleotide substantially identical thereto;
(i) an oligonucleotide consisting of a nucleotide sequence of GAATATAAACTTGTGGTAGTTGGAGCTAT (SEQ ID NO: 9) or an oligonucleotide substantially identical thereto;
(j) an oligonucleotide consisting of a nucleotide sequence of TATAAACTTGTGGTAGTTGGAGGTGT (SEQ ID NO: 10) or an oligonucleotide substantially identical thereto;
(k) an oligonucleotide consisting of a nucleotide sequence of TGAAGATGTACCTATGGTCCTAGTAGGA (SEQ ID NO: 11) or an oligonucleotide substantially identical thereto;
(l) an oligonucleotide consisting of a nucleotide sequence of GTCCTGAGCCTGTTTTGTGTCTA (SEQ ID NO: 12) or an oligonucleotide substantially identical thereto;
(m) a labeled oligonucleotide consisting of a nucleotide sequence of 6FAM-TAGAAGGCAAATCACA (SEQ ID NO: 13) or an oligonucleotide substantially identical thereto;
(n) an oligonucleotide consisting of a nucleotide sequence of TGAATATAAACTTGTGGTAGTTGGAGATA (SEQ ID NO:14) or an oligonucleotide substantially identical thereto;
(o) an oligonucleotide consisting of a nucleotide sequence of AATATAAACTTGTGGTAGTTGGAGGTC (SEQ ID NO:15) or an oligonucleotide substantially identical thereto;
(p) an oligonucleotide consisting of a nucleotide sequence of TGAATATAAACTTGTGGTAGTTGGAGTTT (SEQ ID NO:16) or an oligonucleotide substantially identical thereto;
(q) an oligonucleotide consisting of a nucleotide sequence of AAACTTGTGGTAGTTGGAGCAGA (SEQ ID NO:17) or an oligonucleotide substantially identical thereto;
(r) an oligonucleotide consisting of a nucleotide sequence of AACTTGTGGTAGTTGGAGCAGC (SEQ ID NO:18) or an oligonucleotide substantially identical thereto;
(s) an oligonucleotide consisting of a nucleotide sequence of CACAAAATGATTCTGAATTAGCTGTATC (SEQ ID NO:19) or an oligonucleotide substantially identical thereto; and
(t) a labeled oligonucleotide consisting of 6FAM-TCAAGGCACTCTTGCCT (SEQ ID NO:20) or an oligonucleotide substantially identical thereto.

2. The oligonucleotide of claim 1, selected from:
an oligonucleotide consisting of a nucleotide sequence of GTCAAGGCACTCTTGCCTAAGT (SEQ ID NO: 1) or an oligonucleotide substantially identical thereto;
an oligonucleotide consisting of a nucleotide sequence of GGCCTGCTGAAAATGACTGA (SEQ ID NO:2) or an oligonucleotide substantially identical thereto;
an oligonucleotide consisting of a nucleotide sequence of AGGCACTCTTGCCTCCGT (SEQ ID NO:4) or an oligonucicotidc substantially identical thereto;
an oligonucleotide consisting of a nucleotide sequence of GCCTGCTGAAAATGACTGAATAT (SEQ ID NO:5) or an oligonucleotide substantially identical thereto;
an oligonucleotide consisting of a nucleotide sequence of CTTGTGGTAGTTGGAGCTGGTAA (SEQ ID NO: 7) or an oligonucleotide substantially identical thereto;
an oligonucicotidc consisting of a nucleotide sequence of AATATAAACTTGTGGTAGTTGGAGCTTT (SEQ ID NO: 8) or an oligonucleotide substantially identical thereto;
an oligonucleotide consisting of a nucleotide sequence of GAATATAAACTTGTGGTAGTTGGAGCTAT (SEQ ID NO: 9) or an oligonucleotide substantially identical thereto;
an oligonucleotide consisting of a nucleotide sequence of TATAAACTTGTGGTAGTTGGAGGTGT (SEQ ID NO: 10) or an oligonucleotide substantially identical thereto;
an oligonucleotide consisting of a nucleotide sequence of TGAAGATGTACCTATGGTCCTAGTAGGA (SEQ ID NO: 11) or an oligonucleotide substantially identical thereto;
an oligonucleotide consisting of a nucleotide sequence of GTCCTGAGCCTGTTTTGTGTCTA (SEQ ID NO: 12) or an oligonucleotide substantially identical thereto;
an oligonucleotide consisting of a nucleotide sequence of TGAATATAAACTTGTGGTAGTTGGAGATA (SEQ ID NO:14) or an oligonucicotidc substantially identical thereto;
an oligonucleotide consisting of a nucleotide sequence of AATATAAACTTGTGGTAGTTGGAGGTC (SEQ ID NO:15) or an oligonucleotide substantially identical thereto;
an oligonucleotide consisting of a nucleotide sequence of TGAATATAAACTTGTGGTAGTTGGAGTTT (SEQ ID NO:16) or an oligonucleotide substantially identical thereto;
an oligonucicotidc consisting of a nucleotide sequence of AAACTTGTGGTAGTTGGAGCAGA (SEQ ID NO:17) or an oligonucleotide substantially identical thereto;
an oligonucleotide consisting of a nucleotide sequence of AACTTGTGGTAGTTGGAGCAGC (SEQ ID NO:18) or an oligonucleotide substantially identical thereto; and
an oligonucleotide consisting of a nucleotide sequence of CACAAAATGATTCTGAATTAGCTGTATC (SEQ ID NO:19) or an oligonucleotide substantially identical thereto.

3. The oligonucleotide of claim 2, selected from:
an oligonucleotide consisting of a nucleotide sequence of GTCAAGGCACTCTTGCCTAAGT (SEQ ID NO:1) or an oligonucleotide substantially identical thereto;
an oligonucleotide consisting of a nucleotide sequence of AGGCACTCTTGCCTCCGT (SEQ ID NO:4) or an oligonucleotide substantially identical thereto; and
an oligonucleotide consisting of a nucleotide sequence of CTTGTGGTAGTTGGAGCTGGTAA (SEQ ID NO: 7) or an oligonucleotide substantially identical thereto.

4. The oligonucleotide of claim 2, selected from:
an oligonucleotide consisting of a nucleotide sequence of GGCCTGCTGAAAATGACTGA (SEQ ID NO:2) or an oligonucleotide substantially identical thereto; and
an oligonucleotide consisting of a nucleotide sequence of GCCTGCTGAAAATGACTGAATAT (SEQ ID NO:5) or an oligonucleotide substantially identical thereto.

5. The oligonucleotide of claim 2, selected from:
an oligonucleotide consisting of a nucleotide sequence of AATATAAACTTGTGGTAGTTGGAGCTTT (SEQ ID NO: 8) or an oligonucleotide substantially identical thereto;
an oligonucleotide consisting of a nucleotide sequence of TGAATATAAACTTGTGGTAGTTGGAGATA (SEQ ID NO:14) or an oligonucleotide substantially identical thereto;
an oligonucleotide consisting of a nucleotide sequence of AATATAAACTTGTGGTAGTTGGAGGTC (SEQ ID NO:15) or an oligonucicotidc substantially identical thereto;
an oligonucleotide consisting of a nucleotide sequence of TGAATATAAACTTGTGGTAGTTGGAGTTT (SEQ ID NO:16) or an oligonucleotide substantially identical thereto;
an oligonucleotide consisting of a nucleotide sequence of AAACTTGTGGTAGTTGGAGCAGA (SEQ ID NO:17) or an oligonucleotide substantially identical thereto;
an oligonucicotidc consisting of a nucleotide sequence of AACTTGTGGTAGTTGGAGCAGC (SEQ ID NO:18) or an oligonucleotide substantially identical thereto; and
an oligonucleotide consisting of a nucleotide sequence of CACAAAATGATTCTGAATTAGCTGTATC (SEQ ID NO:19) or an oligonucleotide substantially identical thereto.

6. The oligonucleotide of claim 2 selected from:
an oligonucleotide consisting of a nucleotide sequence of GAATATAAACTTGTGGTAGTTGGAGCTAT (SEQ ID NO: 9) or an oligonucleotide substantially identical thereto; and
an oligonucleotide consisting of a nucleotide sequence of TATAAACTTGTGGTAGTTGGAGGTGT (SEQ ID NO: 10) or an oligonucleotide substantially identical thereto.

7. The oligonucleotide of claim 2 selected from:
an oligonucleotide consisting of a nucleotide sequence of TGAAGATGTACCTATGGTCCTAGTAGGA (SEQ ID NO: 11) or an oligonucleotide substantially identical thereto; and
an oligonucleotide consisting of a nucleotide sequence of GTCCTGAGCCTGTTTTGTGTCTA (SEQ ID NO: 12) or an oligonucleotide substantially identical thereto.

8. A kit comprising at least one of the oligonucleotides (a) through (t) according to claim 1.

9. The kit of claim 8, comprising at least one of the oligonucleotides (a), (b), (d), (e), (g), (h), (i), (j), (k), (l), (n), (o), (p), (q), (r) and (s), and at least one of the oligonucleotides (c), (f), (m) and (t).

10. A method of detecting a KRAS mutation in DNA, comprising:
(1) amplifying the DNA with PCR using a thermostable DNA polymerase lacking 3′ exonuclease activity and
(I) a pair of control oligonucleotide primers for a control assay, wherein the pair of control oligonucleotide primers are for amplification of a DNA region in exon 4 of the KRAS gene, and wherein the pair of control oligonucleotide primers are KrasEx4 Control Forward Primer consisting of the nucleotide sequence represented by SEQ ID NO:11 or an oligonucleotide substantially identical thereto according to claim 1, and KrasEx4 Control Reverse Primer consisting of the nucleotide sequence represented by SEQ ID NO:12 or an oligonucleotide substantially identical thereto according to claim 1; and
(II) at least one pair of mutant oligonucleotide primers for mutation assay, wherein the at least one pair of mutant oligonucleotide primers are for amplification of a DNA region having a mutation in codon 12 and/or a mutation in codon 13 located in exon 2 of the KRAS gene, and wherein the at least one pair of mutant oligonucleotide primers are selected from
(A) a first pair of codon 13 mutant oligonucleotide primers having
(i) a reverse primer selected from (a) 13ASP Reverse Primer consisting of the nucleotide sequence represented by SEQ ID NO:1 or an oligonucleotide substantially identical thereto according to claim 1, or (b) an oligonucleotide consisting of the nucleotide sequence represented by SEQ ID NO:4 (Kras38A_{—}3TG-R) or an oligonucleotide substantially identical thereto according to claim 1, and
(ii) a forward primer selected from (a) C13 Forward Primer consisting of the nucleotide sequence represented by SEQ ID NO:2 or an oligonucleotide substantially identical thereto according to claim 1, or (b) an oligonucleotide consisting of the nucleotide sequence represented by SEQ ID NO:5 (KrasC13-F) or an oligonucleotide substantially identical thereto according to claim 1;
(B) a second pair of codon 13 mutant oligonucleotide primers having
(i) a forward primer consisting of the nucleotide sequence represented by SEQ ID NO:7 (13ASP Forward Primer) or an oligonucleotide substantially identical thereto according to claim 1; and
(ii) a reverse primer consisting of the nucleotide sequence represented by SEQ ID NO:19 (C12 Common Reverse Primer) or an oligonucleotide substantially identical thereto according to claim 1; or
(C) at least one pair of codon 12 mutant oligonucleotide primers comprising
(i) at least one forward primer selected from (a) an oligonucleotide consisting of the nucleotide sequence represented by SEQ ID NO:8 (12VAL Forward Primer) or an oligonucleotide substantially identical thereto according to claim 1; (b) an oligonucleotide consisting of the nucleotide sequence represented by SEQ ID NO:14 (12SER Forward Primer) or an oligonucleotide substantially identical thereto according to claim 1; (c) an oligonucleotide consisting of the nucleotide sequence represented by SEQ ID NO:15 (12ARG Forward Primer) or an oligonucleotide substantially identical thereto according to claim 1; (d) an oligonucleotide consisting of the nucleotide sequence represented by SEQ ID NO:16 (12CYS Forward Primer) or an oligonucleotide substantially identical thereto according to claim 1; (e) an oligonucleotide consisting of the nucleotide sequence represented by SEQ ID NO:17 (12ASP Forward Primer) or an oligonucleotide substantially identical thereto according to claim 1; (f) an oligonucleotide consisting of the nucleotide sequence represented by SEQ ID NO:18 (12ALA Forward Primer) or an oligonucleotide substantially identical thereto according to claim 1; (g) an oligonucleotide consisting of the nucleotide sequence represented by SEQ ID NO:9 (KrasM35T_{—}1GA-F) or an oligonucleotide substantially identical thereto according to claim 1; or (h) an oligonucleotide consisting of the nucleotide sequence represented by SEQ ID NO:10 (Kras35T_{—}3CG-F) or an oligonucleotide substantially identical thereto according to claim 1; and
(ii) an oligonucleotide reverse primer consisting of a nucleotide sequence represented by SEQ ID NO:19 (the C12 Common Reverse Primer) or an oligonucleotide substantially identical thereto according to claim 1;
(2) determining the product of step (1)(I) comprising an amplification product of the DNA region of exon 4 amplified by the pair of control oligonucleotide primers, wherein the detection of the amplification product indicates the presence of the KRAS gene in the DNA; and
(3) determining the product of step (1)(II) comprising an amplification product of the DNA region of exon 2 amplified by the pair of mutant oligonucleotide primers, wherein
(a) the detection of the amplification product when at least one pair of codon 13 mutant oligonucleotide primers is used in step (1)(II) indicates the presence of a mutation in codon 13 in exon 2 of the KRAS gene in the DNA; and/or
(b) the detection of the amplification product when at least one pair of codon 12 mutant oligonucicotidc primers is used in step (1)(II) indicates the presence of a mutation in codon 12 in exon 2 of the KRAS gene in the DNA.

11. The method of claim 10, wherein in step (1)(II), the at least one pair of mutant oligonucleotide primers used in step (1)(II) are for amplification of the DNA region having a mutation in codon 12 located in exon 2 of the KRAS gene, the at least one pair of mutant oligonucleotide primers for codon 12 comprising
(i) at least one forward primer selected from (a) an oligonucleotide consisting of the nucleotide sequence represented by SEQ ID NO:8 (12VAL Forward Primer) or an oligonucleotide substantially identical thereto according to claim 1; (b) an oligonucleotide consisting of the nucleotide sequence represented by SEQ ID NO:14 (12SER Forward Primer) or an oligonucleotide substantially identical thereto according to claim 1; (c) an oligonucleotide consisting of the nucleotide sequence represented by SEQ ID NO:15 (12ARG Forward Primer) or an oligonucicotidc substantially identical thereto according to claim 1; (d) an oligonucleotide consisting of the nucleotide sequence represented by SEQ ID NO:16 (12CYS Forward Primer) or an oligonucleotide substantially identical thereto according to claim 1; (e) an oligonucleotide consisting of the nucleotide sequence represented by SEQ ID NO:17 (12ASP Forward Primer) or an oligonucleotide substantially identical thereto according to claim 1; (f) an oligonucleotide consisting of the nucleotide sequence represented by SEQ ID NO:18 (12ALA Forward Primer) or an oligonucleotide substantially identical thereto according to claim 1; (g) an oligonucleotide consisting of the nucleotide sequence represented by SEQ ID NO:9 (KrasM35T_{—}1GA-F) or an oligonucleotide substantially identical thereto according to claim 1; or (h) an oligonucleotide consisting of the nucleotide sequence represented by SEQ ID NO:10 (Kras35T_{—}3CG-F) or an oligonucleotide substantially identical thereto according to claim 1; and
(ii) an oligonucleotide reverse primer consisting of a nucleotide sequence represented by SEQ ID NO:19 (the C12 Common Reverse Primer) or an oligonucleotide substantially identical thereto according to claim 1.

12. The method of claim 10, wherein in step (1)(II), the at least one pair of mutant oligonucleotide primers used in step (1)(II) are for amplification of the DNA region having a mutation in codon 13 located in exon 2 of the KRAS gene, the at least one pair of mutant oligonucleotide primers for codon 13 is selected from
(A) a first pair of codon 13 mutant oligonucleotide primers comprising
(i) a reverse primer selected from (a) 13ASP Reverse Primer consisting of the nucleotide sequence represented by SEQ ID NO:1 or an oligonucleotide substantially identical thereto according to claim 1, or (b) an oligonucleotide consisting of the nucleotide sequence represented by SEQ ID NO:4 (Kras38A_{—}3TG-R) or an oligonucleotide substantially identical thereto, and
(ii) a forward primer selected from (a) C13 Forward Primer consisting of the nucleotide sequence represented by SEQ ID NO:2 or an oligonucleotide substantially identical thereto according to claim 1, or (b) an oligonucleotide consisting of the nucleotide sequence represented by SEQ ID NO:5 (KrasC13-F) or an oligonucleotide substantially identical thereto; and
(B) a second pair of codon 13 mutant oligonucleotide primers comprising
(i) a forward primer consisting of the nucleotide sequence represented by SEQ ID NO:7 (13ASP Forward Primer) or an oligonucleotide substantially identical thereto; and
(ii) a reverse primer consisting of the nucleotide sequence represented by SEQ ID NO:19 (C12 Common Reverse Primer) or an oligonucleotide substantially identical thereto.

13. The method of claim 12, wherein in step (1)(II), the at least one pair of mutant oligonucleotide primers comprises
(i) a reverse primer selected from (a) 13ASP Reverse Primer consisting of the nucleotide sequence represented by SEQ ID NO:1 or an oligonucleotide substantially identical thereto according to claim 1, or (b) an oligonucleotide consisting of the nucleotide sequence represented by SEQ ID NO:4 (Kras38A_{—}3TG-R) or an oligonucleotide substantially identical thereto, and
(ii) a forward primer selected from (a) C13 Forward Primer consisting of the nucleotide sequence represented by SEQ ID NO:2 or an oligonucleotide substantially identical thereto according to claim 1, or (b) an oligonucleotide consisting of the nucleotide sequence represented by SEQ ID NO:5 (KrasC13-F) or an oligonucleotide substantially identical thereto.

14. The method of claim 13, wherein the at least one pair of mutant oligonucleotide primers used in step (1)(II) comprises
(i) a 13ASP Reverse Primer consisting of the nucleotide sequence represented by SEQ ID NO: 1 or an oligonucleotide substantially identical thereto, and
(ii) a C13 Forward Primer consisting of the nucleotide sequence represented by SEQ ID NO:2 or an oligonucleotide substantially identical thereto.

15. The method of claim 13, wherein the at least one pair of mutant oligonucleotide primers used in step (1)(II) comprises
(i) a reverse primer oligonucleotide consisting of the nucleotide sequence represented by SEQ ID NO:4 (Kras38A_{—}3TG-R) or an oligonucleotide substantially identical thereto, and
(ii) a forward primer an oligonucleotide consisting of the nucleotide sequence represented by SEQ ID NO:5 (KrasC13-F) or an oligonucleotide substantially identical thereto.

16. The method of claim 11, wherein in step (1)(II), the at least one pair of mutant oligonucleotide primers for codon 13 comprises
(i) a forward primer consisting of the nucleotide sequence represented by SEQ ID NO:7 (13ASP Forward Primer) or an oligonucleotide substantially identical thereto; and
(ii) a reverse primer consisting of the nucleotide sequence represented by SEQ ID NO:19 (C12 Common Reverse Primer) or an oligonucleotide substantially identical thereto.

17. The method of claim 10, wherein in step (2), the amplification product of the DNA region of exon 4 amplified by the pair of control oligonucleotide primers consists of the DNA region spanning from one member of the pair of control oligonucleotide primers to the other member of the pair of control oligonucleotide primers, or spanning from a region complementary to one member of the pair of control oligonucleotide primers to a region complementary to the other member of the pair of control oligonucleotide primers.

18. The method of claim 10, wherein in step (3), the amplification product of the DNA region of exon 2 amplified by the pair of mutant oligonucleotide primers consists of the DNA region spanning from one member of the at least one pair of mutant oligonucleotide primers to the other member of the at least one pair of mutant oligonucleotide primers, or spanning from a region complementary to one member of the at least one pair of mutant oligonucleotide primers to a region complementary to the other member of the at least one pair of mutant oligonucleotide primers.

19. The method of claim 10, wherein the thermostable DNA polymerase lacking 3′ exonuclease activity used in step (1) is selected from thermostable Bst DNA polymerase I isolated from Bacillus stearothermophilus, IsoTherm DNA polymerase, T7 DNA polymerase having the 3′ to 5′ exonuclease activity removed via oxidation of the amino acid residues essential for the exonuclease activity (Sequenase Vertion 1) or genetically by deleting 28 amino acids essential for the 3′ to 5′ exonuclease activity (Sequenase Version 2), Vent_{R}(exo⁻) DNA polymerase and Taq polymerase.

20. The method of claim 19, wherein the thermostable DNA polymerase lacking 3′ exonuclease activity is Taq polymerase.

21. The method of claim 17, wherein in step (2), the amplification product of the DNA region of exon 4 amplified by the pair of control oligonucleotide primers is determined with an oligonucleotide probe consisting of the nucleotide sequence represented by SEQ ID NO:13, or a labeled oligonucleotide substantially identical thereto.

22. The method of claim 18, wherein in step (2), the amplification product of the DNA region of exon 2 amplified by the at least one pair of mutant oligonucleotide primers is determined with an oligonucleotide probe consisting of the nucleotide sequence represented by SEQ ID NO:3 or 6, or a labeled oligonucleotide substantially identical thereto.

23. The method of claim 22, wherein in step (2), the amplification product of the DNA region of exon 2 amplified by the at least one pair of mutant oligonucleotide primers is determined with an oligonucleotide probe consisting of the nucleotide sequence represented by SEQ ID NO:3, or a labeled oligonucleotide substantially identical thereto.

24. The method of claim 22, wherein in step (2), the amplification product of the DNA region of exon 2 amplified by the at least one pair of mutant oligonucleotide primers is determined with an oligonucleotide probe consisting of the nucleotide sequence represented by SEQ ID NO:6, or a labeled oligonucleotide substantially identical thereto.

25. The method of claim 14, wherein in step (2), the amplification product of the DNA region of exon 2 amplified by the at least one pair of mutant oligonucleotide primers is determined with an oligonucleotide probe consisting of the nucleotide sequence represented by SEQ ID NO:3, or a labeled oligonucleotide substantially identical thereto.

26. The method of claim 15, wherein in step (2), the amplification product of the DNA region of exon 2 amplified by the at least one pair of mutant oligonucleotide primers is determined with an oligonucleotide probe consisting of the nucleotide sequence represented by SEQ ID NO:6, or a labeled oligonucleotide substantially identical thereto.

27. The method of claim 1, wherein the DNA used in step (1) is genomic DNA or cDNA obtained from a tissue.

28. The method of claim 27, wherein the DNA used in step (1) is cDNA obtained from a tissue.

29. A method of predicting the sensitivity of a tumor in a patient to epidermal growth factor receptor-directed chemotherapy, comprising
(1) obtaining DNA from the tumor; and
(2) determining the presence of a mutation in codon 12 and/or a mutation in codon 13 in exon 2 of the KRAS gene in the DNA using the method of one of claims 10-28 for detecting a KRAS mutation in DNA, wherein the detection of the mutation in codon 12 and/or a mutation in codon 13 predicts that the tumor has reduced sensitivity toward epidermal growth factor receptor-directed chemotherapy compared with tumors of the same type having no mutation in codon 12 and codon 13.

30. The method of claim 29, wherein step (2) determines a mutation in codon 12 in exon 2 of the KRAS gene.

31. The method of claim 29, wherein step (2) determines a mutation in codon 13 in exon 2 of the KRAS gene.

32. The method of claim 29, wherein step (2) determines a mutation in codon 12 and a mutation in codon 13 in exon 2 of the KRAS gene.


SEQUENCE LISTING (62 sequences)

SEQ ID NO: 1 - DNA, 22 bp, Artificial Sequence
  Primer
gtcaaggcactcttgcctaagt

SEQ ID NO: 2 - DNA, 20 bp, Artificial Sequence
  Primer
ggcctgctgaaaatgactga

SEQ ID NO: 3 - DNA, 16 bp, Artificial Sequence
  Probe
caactaccacaagttt

SEQ ID NO: 4 - DNA, 18 bp, Artificial Sequence
  Primer
aggcactcttgcctccgt

SEQ ID NO: 5 - DNA, 23 bp, Artificial Sequence
  Primer
gcctgctgaaaatgactgaatat

SEQ ID NO: 6 - DNA, 18 bp, Artificial Sequence
  Probe
ctccaactaccacaagtt

SEQ ID NO: 7 - DNA, 23 bp, Artificial Sequence
  Primer
cttgtggtagttggagctggtaa

SEQ ID NO: 8 - DNA, 28 bp, Artificial Sequence
  Primer
aatataaacttgtggtagttggagcttt

SEQ ID NO: 9 - DNA, 29 bp, Artificial Sequence
  Primer
gaatataaacttgtggtagttggagctat

SEQ ID NO: 10 - DNA, 26 bp, Artificial Sequence
  Primer
tataaacttgtggtagttggaggtgt

SEQ ID NO: 11 - DNA, 28 bp, Artificial Sequence
  Primer
tgaagatgtacctatggtcctagtagga

SEQ ID NO: 12 - DNA, 23 bp, Artificial Sequence
  Primer
gtcctgagcctgttttgtgtcta

SEQ ID NO: 13 - DNA, 16 bp, Artificial Sequence
  Probe
tagaaggcaaatcaca

SEQ ID NO: 14 - DNA, 29 bp, Artificial Sequence
  Primer
tgaatataaacttgtggtagttggagata

SEQ ID NO: 15 - DNA, 27 bp, Artificial Sequence
  Primer
aatataaacttgtggtagttggaggtc

SEQ ID NO: 16 - DNA, 29 bp, Artificial Sequence
  Primer
tgaatataaacttgtggtagttggagttt

SEQ ID NO: 17 - DNA, 23 bp, Artificial Sequence
  Primer
aaacttgtggtagttggagcaga

SEQ ID NO: 18 - DNA, 22 bp, Artificial Sequence
  Primer
aacttgtggtagttggagcagc

SEQ ID NO: 19 - DNA, 28 bp, Artificial Sequence
  Primer
cacaaaatgattctgaattagctgtatc

SEQ ID NO: 20 - DNA, 17 bp, Artificial Sequence
  Probe
tcaaggcactcttgcct

SEQ ID NO: 21 - DNA, 23 bp, Artificial Sequence
  Primer
cgtcaaggcactcttgcctaagt

SEQ ID NO: 22 - DNA, 24 bp, Artificial Sequence
  Primer
tcgtcaaggcactcttgcctaagt

SEQ ID NO: 23 - DNA, 25 bp, Artificial Sequence
  Primer
atcgtcaaggcactcttgcctaagt

SEQ ID NO: 24 - DNA, 21 bp, Artificial Sequence
  Primer
aggcctgctgaaaatgactga

SEQ ID NO: 25 - DNA, 22 bp, Artificial Sequence
  Primer
aaggcctgctgaaaatgactga

SEQ ID NO: 26 - DNA, 23 bp, Artificial Sequence
  Primer
taaggcctgctgaaaatgactga

SEQ ID NO: 27 - DNA, 19 bp, Artificial Sequence
  Primer
aaggcactcttgcctccgt

SEQ ID NO: 28 - DNA, 20 bp, Artificial Sequence
  Primer
caaggcactcttgcctccgt

SEQ ID NO: 29 - DNA, 21 bp, Artificial Sequence
  Primer
tcaaggcactcttgcctccgt

SEQ ID NO: 30 - DNA, 24 bp, Artificial Sequence
  Primer
ggcctgctgaaaatgactgaatat

SEQ ID NO: 31 - DNA, 25 bp, Artificial Sequence
  Primer
aggcctgctgaaaatgactgaatat

SEQ ID NO: 32 - DNA, 26 bp, Artificial Sequence
  Primer
aaggcctgctgaaaatgactgaatat

SEQ ID NO: 33 - DNA, 24 bp, Artificial Sequence
  Primer
acttgtggtagttggagctggtaa

SEQ ID NO: 34 - DNA, 25 bp, Artificial Sequence
  Primer
aacttgtggtagttggagctggtaa

SEQ ID NO: 35 - DNA, 26 bp, Artificial Sequence
  Primer
aaacttgtggtagttggagctggtaa

SEQ ID NO: 36 - DNA, 29 bp, Artificial Sequence
  Primer
gaatataaacttgtggtagttggagcttt

SEQ ID NO: 37 - DNA, 30 bp, Artificial Sequence
  Primer
tgaatataaacttgtggtagttggagcttt

SEQ ID NO: 38 - DNA, 31 bp, Artificial Sequence
  Primer
ctgaatataaacttgtggtagttggagcttt

SEQ ID NO: 39 - DNA, 30 bp, Artificial Sequence
  Primer
tgaatataaacttgtggtagttggagctat

SEQ ID NO: 40 - DNA, 31 bp, Artificial Sequence
  Primer
ctgaatataaacttgtggtagttggagctat

SEQ ID NO: 41 - DNA, 32 bp, Artificial Sequence
  Primer
actgaatataaacttgtggtagttggagctat

SEQ ID NO: 42 - DNA, 27 bp, Artificial Sequence
  Primer
atataaacttgtggtagttggaggtgt

SEQ ID NO: 43 - DNA, 28 bp, Artificial Sequence
  Primer
aatataaacttgtggtagttggaggtgt

SEQ ID NO: 44 - DNA, 29 bp, Artificial Sequence
  Primer
gaatataaacttgtggtagttggaggtgt

SEQ ID NO: 45 - DNA, 30 bp, Artificial Sequence
  Primer
ctgaatataaacttgtggtagttggagata

SEQ ID NO: 46 - DNA, 31 bp, Artificial Sequence
  Primer
actgaatataaacttgtggtagttggagata

SEQ ID NO: 47 - DNA, 32 bp, Artificial Sequence
  Primer
gactgaatataaacttgtggtagttggagata

SEQ ID NO: 48 - DNA, 28 bp, Artificial Sequence
  Primer
gaatataaacttgtggtagttggaggtc

SEQ ID NO: 49 - DNA, 29 bp, Artificial Sequence
  Primer
tgaatataaacttgtggtagttggaggtc

SEQ ID NO: 50 - DNA, 30 bp, Artificial Sequence
  Primer
ctgaatataaacttgtggtagttggaggtc

SEQ ID NO: 51 - DNA, 30 bp, Artificial Sequence
  Primer
ctgaatataaacttgtggtagttggagttt

SEQ ID NO: 52 - DNA, 31 bp, Artificial Sequence
  Primer
actgaatataaacttgtggtagttggagttt

SEQ ID NO: 53 - DNA, 32 bp, Artificial Sequence
  Primer
gactgaatataaacttgtggtagttggagttt

SEQ ID NO: 54 - DNA, 24 bp, Artificial Sequence
  Primer
taaacttgtggtagttggagcaga

SEQ ID NO: 55 - DNA, 25 bp, Artificial Sequence
  Primer
ataaacttgtggtagttggagcaga

SEQ ID NO: 56 - DNA, 26 bp, Artificial Sequence
  Primer
tataaacttgtggtagttggagcaga

SEQ ID NO: 57 - DNA, 23 bp, Artificial Sequence
  Primer
aaacttgtggtagttggagcagc

SEQ ID NO: 58 - DNA, 24 bp, Artificial Sequence
  Primer
taaacttgtggtagttggagcagc

SEQ ID NO: 59 - DNA, 25 bp, Artificial Sequence
  Primer
ataaacttgtggtagttggagcagc

SEQ ID NO: 60 - DNA, 29 bp, Artificial Sequence
  Primer
ccacaaaatgattctgaattagctgtatc

SEQ ID NO: 61 - DNA, 30 bp, Artificial Sequence
  Primer
tccacaaaatgattctgaattagctgtatc

SEQ ID NO: 62 - DNA, 31 bp, Artificial Sequence
  Primer
gtccacaaaatgattctgaattagctgtatc